(19) United States
(12) Patent Application Publication
(10) Pub. No.: US 2020/0129495 A1
(43) Pub. Date: Apr. 30, 2020

(54) EBASTINE TOPICAL COMPOSITION

(71) Applicant: BioPharmX, Inc., Menlo Park, CA (US)

(72) Inventors: Xin CHEN, Palo Alto, CA (US); Mouhannad JUMAA, San Jose, CA (US); Usha NAGAVARAPU, San Jose, CA (US); Maiko C. HERMSMEIER, San Jose, CA (US)

(21) Appl. No.: 16/174,216

(22) Filed: Oct. 29, 2018

(51) Int. Cl. A61K 31/4515; A61K 9/00; A61P 17/14; A61K 47/10

(52) U.S. Cl. CPC A61K 31/4515; A61K 47/10; A61P 17/14; A61K 9/0014

(57) ABSTRACT

Provided herein is a topical composition for treatment of a hair-loss related condition or disease and related methods for making and using the topical composition. The topical composition generally comprises ebastine dissolved in a solvent. In some exemplary embodiments, the solvent comprises a monohydric aliphatic alcohol and an ester. In some exemplary embodiments, the solvent comprises a polyol.

### TECHNICAL FIELD

This disclosure relates generally to a topical pharmaceutical composition comprising ebastine, to methods for preparing the composition and to methods of using the composition. More particularly, this disclosure is directed to a stable topical composition comprising ebastine dissolved in a solvent, and to methods for making and using such a composition, such as, for example, methods of treatment for a hair-loss related condition or disease.

### BACKGROUND

Hair-loss related conditions or diseases, although not life-threatening, cause emotional distress due to problems related to appearance. Therefore, the promotion of hair growth in the treatment for hair-loss related conditions or diseases is desirable.

Alopecia is a disease, which involves hair loss. There are a number of different types of alopecia whose symptoms vary depending on the cause of the condition. The most common form of alopecia is androgenetic alopecia (AGA). AGA is a type of alopecia in which hormones act on hormone-sensitive hair follicles to replace normal hair growth with smaller, rapid-cycling vellus hairs. AGA occurs in both males and females, occurring in about 50 percent of males and 10 to 20 percent of females.

An approach for promoting new hair growth in the treatment of a hair-loss related condition or disease involves chemical treatment. With regard to AGA in particular, existing chemical treatments include, for example, minoxidil and antiandrogens, such as finasteride. While these treatments are reasonably effective in preventing or delaying hair loss, they present challenges. Minoxidil is accompanied by negative side effects such as decreased blood pressure and decreased heart rate. These symptoms recur when use of minoxidil has stopped. Finasteride is accompanied by negative side effects such as prostate hyperplasia, erectile dysfunction, and ejaculatory disorder. Again, these symptoms recur when use of finasteride has stopped.

There remains a need for additional topical compositions and treatment methods for promoting and maintaining hair growth in the treatment of hair-loss related conditions or diseases and that do not elicit the same side effect profile as minoxidil and finasteride and can effectively be applied topically.

### BRIEF SUMMARY

The following aspects and embodiments thereof described and illustrated below are exemplary and illustrative, and should not be read as limiting in scope.

In a first aspect, provided is a topical pharmaceutical composition comprising ebastine dissolved in a solvent.

In some embodiments, the solvent is non-aqueous.

In some embodiments, the solvent comprises a volatile solvent and a non-volatile solvent. In some embodiments, loss of substantially all of the volatile solvent from the solvent creates a residual composition in which ebastine is dissolved. In some embodiments, ebastine is more soluble at room temperature in the volatile solvent than in the non-volatile solvent. In some embodiments, ebastine is more soluble at room temperature in the non-volatile solvent than in the volatile solvent. In some embodiments, ebastine is more soluble at room temperature in the solvent than in either the volatile solvent or the non-volatile solvent. In some embodiments, the solubility of ebastine in the solvent at room temperature in the solvent exceeds by at least 3%, 5%, 7%, 10%, 15%, 20%, or 25% the solubility of ebastine in a reference solvent, such as a component of the solvent, e.g., the volatile solvent or the non-volatile solvent if the solvent comprises a volatile or non-volatile solvent.

In some embodiments, the volatile solvent is a monohydric aliphatic alcohol and the non-volatile solvent is an ester. In some embodiments, the monohydric aliphatic alcohol is ethanol. In further embodiments, the ester is diethyl sebacate or ethyl acetate or dimethyl succinate.

In some embodiments, the concentration of ebastine in the composition is in the range of 0.1 percent w/w to 5 percent w/w. In some embodiments, the concentration of ebastine in the composition is about 2.0 percent w/w.

In some embodiments, the w/w ratio of monohydric aliphatic alcohol to ester is in the range of 1:1 to 99:1. In some embodiments, the w/w ratio of monohydric aliphatic alcohol to ester is in the range of 1:1 to 20:1. In some embodiments the monohydric aliphatic alcohol comprises 40 percent to 99 percent of the composition by weight.

In some embodiments, the w/w ratio of the volatile solvent to the non-volatile solvent is between about 1:1 to 99:1. In some embodiments, the w/w ratio of the volatile solvent to the non-volatile solvent is in the range of 1:1 to 20:1.

In some embodiments, the topical pharmaceutical composition further comprises a polyol. In some embodiments, the polyol is a C3-C8 diol or a triol. In some embodiments, the polyol is propylene glycol.

In some embodiments, the ebastine in the topical pharmaceutical composition is stable when stored in a sealed glass container for at least 2 months or longer at 40° C. in a dark environment.

In some embodiments, the topical pharmaceutical composition further comprises water. In some embodiments, the concentration by weight of the non-aqueous solvent exceeds the concentration by weight of water. In some embodiments, the composition has a pH in the range of about 3 to about 9 or of 3 to 8 or of 3 to 7 or of 3 to 6.

In some embodiments, the composition does not comprise an active pharmaceutical ingredient other than ebastine.

In a second aspect, provided is a method for treatment of a hair-loss related condition or disease comprising topically applying a composition comprising ebastine dissolved in a solvent to a target surface of a mammalian body at least once daily for a period of at least one month.

In some embodiments, the hair-loss related condition or disease is selected from the group consisting of scarring alopecia, non-scarring alopecia, androgenetic alopecia, and alopecia areata.

In some embodiments, the solvent is non-aqueous. In some embodiments, the solvent is comprises a volatile solvent and a non-volatile solvent. In some embodiments, the volatile solvent concentration in the composition is wholly or partially reduced by evaporation to create a residual composition that remains on the affected area for a treatment period.

In some embodiments, the target surface is the scalp. In some embodiments, the disease is androgenetic alopecia. In some embodiments, the daily dosage of ebastine is in the range of 1.0 mg/cm² and 10 mg/cm². In some embodiments, the daily dosage of ebastine is about 2.5 mg/cm².

In some embodiments, the composition does not comprise an active pharmaceutical ingredient other than ebastine.

In a third aspect, provided is a method for treatment of a dermatological disorder that benefits from antihistamine activity, such as, for example, contact dermatitis, seborrheic dermatitis, or atopic dermatitis, including eczema, the method comprising topically applying a composition comprising ebastine dissolved in a solvent to a target surface of a mammalian body at least once daily for a period of at least one week.

In a fourth aspect, provided is a method for preparing a topical pharmaceutical composition, the method comprising (i) combining ebastine in a solvent to form a mixture and (ii) agitating the mixture from (i) to form a solution in which the ebastine is dissolved.

In yet another aspect, a kit for the delivery of a topical pharmaceutical composition is provided. The kit comprises a container closure, a topical pharmaceutical composition comprising ebastine dissolved in a solvent, and instructions for use, wherein instructions for use comprises instructions to apply the composition to a skin surface, such as the scalp, of a patient.

In some embodiments, the container closure comprises a nozzle capable of releasing the composition. In some embodiments, the instructions further specify applying the composition to the skin surface once daily.

### BRIEF DESCRIPTION OF THE DRAWINGS

FIG. 1 is a graph illustrating the relative concentrations of ebastine over time in compositions comprising ebastine and ethanol, as described in Example 2. The degradation and stability of the ebastine composition was measured at 1-day, 1-week, 2-week, 3-week, and 2-month time points following storage in a 40° C. oven within sealed clear glass vials.

FIG. 2 is a graph illustrating the solubility of ebastine in water. Ebastine concentrations were recorded at pH values within the range from 3 to 7 as described in Example 3.

FIG. 3 is a graph illustrating the solubility of ebastine in a 90:10 ethanol:water solvent system. Ebastine concentrations were recorded at pH values within the range from 2 to 10 as described in Example 4.

FIG. 4 is a graph illustrating the relative concentrations of ebastine over time in compositions comprising ebastine, ethanol, and water at a pH range from 2 to 6 as described in Example 5. The degradation and stability of the ebastine composition were measured at 1-day, 1-week, 1-month, 2-month, and 3-month time points following storage in a 40° C. oven within sealed clear glass vials.

FIGS. 5A-5C are graphs which illustrate the solubility of ebastine in compositions comprising ethanol and selected esters as described in Example 6. Mixtures comprising esters and ethanol show enhanced solubility when compared to individual solvent components.

FIG. 6 is a graph illustrating the relative concentrations of ebastine over time in exemplary compositions described in Example 7 and Table 7. The degradation and stability of the ebastine composition was measured at 1-day, 1-week, 4-week, and 8-week time points following storage in a 40° C. oven within sealed clear glass vials.

FIG. 7 is a graph that compares the average length of hairs for the patch area of mice treated daily with topical ebastine composition, the average length of hairs for the patch area of mice that received no topical treatment, and the average length of hairs for the non-patch area of mice that received no topical treatment. Data presented was collected at study end after 5 weeks of study, as described in Example 9. Hair length was measured only for observed hairs and this measurement does not account for the proportion of the patch that was not covered by hair growth. Values above the bars represent the percentage of the patch covered by the hairs measured.

FIG. 8 is a graph illustrating the average percent of the patch test area covered with hair of mice treated with topical ebastine composition and non-treated mice, as described in Example 9. Duration of the study period was 5 weeks and evaluations were made weekly.

FIGS. 9A-9B are photographs which illustrate hair growth over a 5-week study period for a non-treated mouse (FIG. 9A) and for a mouse treated with the topical ebastine composition (FIG. 9B) as described in Example 9.

### DETAILED DESCRIPTION

The present invention will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As can be appreciated from the foregoing and following description, each and every feature described herein, and each and every combination of two or more of such features, is included within the scope of the present disclosure provided that the features included in that such combinations are not inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

All publications, patents and patent applications cited herein are hereby incorporated by reference in their entirety, unless otherwise indicated. In an instance in which the same term is defined both in a publication, patent, or patent application incorporated herein by reference and in the present disclosure, the definition in the present disclosure represents the controlling definition. For publications, patents, and patent applications referenced for their description of a particular type of compound, chemistry, etc., portions pertaining to such compounds, chemistry, etc. are those portions of the document that are incorporated herein by reference.

### Definitions

It must be noted that, as used in this specification, the singular forms “a,” “an,” and “the” include plural referents unless the context clearly dictates otherwise. Thus, for example, reference to an “active pharmaceutical ingredient” includes a single ingredient as well as two or more different ingredients, reference to a “solvent” refers to a single solvent as well as to two or more different solvents.

In describing and claiming the present invention, the following terminology will be used in accordance with the definitions described below.

The term “topical composition” refers to a material that comprises pharmaceutically acceptable ingredients, including an active pharmaceutical ingredient, and is intended for administration to a mammal or human patient by application to the surface of the skin.

The term “treatment of a hair-loss related condition or disease” refers to alleviation of symptoms associated with a hair-loss related condition or disease, treatment of a hair-loss related condition or disease, prophylaxis of a hair-loss related condition or disease, and/or prevention of a hair-loss related condition or disease.

As used herein, “hair-loss related condition or disease” refers to undesirable hair thinning or hair loss by a patient. Hair-loss related conditions and diseases include but are not limited to scarring alopecia, non scarring alopecia, androgenetic alopecia, and alopecia areata. A hair-loss related condition or disease may be caused by, for example, topical inflammatory skin conditions.

Ebastine may be soluble to different degrees in different solvents. Ebastine, when measured in a solvent at 25 degrees C. and atmospheric pressure as described in Example 1, is said to be “soluble” in a solvent if it has a solubility of 33.3 mg/g or greater in that solvent, “sparingly soluble” if it has a solubility of 10.0 to 33.3 mg/g, “at least sparingly soluble” if it has a solubility of 10.0 mg/g or greater, “slightly soluble” if it has a solubility of 1.0 to 10.0 mg/g, and “insoluble” if it has a solubility of less than 1.0 mg/g.

The term “solvent” refers to a substance, usually a liquid, in which ebastine is at least sparingly soluble.

The term “ebastine” refers to 1-[4-(1,1-dimethylethyl)phenyl]-4-[4-(diphenylmethoxy)-1-piperidinyl]-1-butanone (i.e. CAS number 90729-43-4), pharmaceutically acceptable salt forms thereof, solvates thereof, and hydrates thereof.

The term “monohydric aliphatic alcohol” and “alcohol” are used interchangeably to refer to a monofunctional organic compound that contains a single hydroxyl group, in which the hydroxyl functional group is covalently attached to a saturated carbon atom forming part of a branched or linear alkyl chain, and which does not contain an aromatic-ring configuration of atoms. Generally, a monohydric aliphatic alcohol conforms to the formula R—OH, where R is a C₁-C₄ alkyl group. Suitable R groups include methyl, ethyl, propyl, isopropyl, butyl, sec-butyl, isobutyl and tert-butyl.

The term “polyol” refers to a pharmaceutically acceptable alcohol containing two or more hydroxyl groups, and possessing from 3-8 carbon atoms.

The term “topical”, in reference to administration of a drug or composition, refers to application of such drug or composition to an exterior epithelial surface of the body, including the skin or cornea. For purposes of this application, applications inside a bodily orifice, such as the mouth, nose, or ear shall not be considered to be topical applications.

A solvent (or solvents) is said to “dissolve” ebastine (or conversely, ebastine is said to be dissolved in a solvent) if the solubility of ebastine, as measured in Example 1, at 25 degrees centigrade and atmospheric pressure, is greater than the concentration of the drug in solvent.

A solvent or composition is said to be “volatile” if it has a boiling point of less than 100 degrees centigrade at atmospheric pressure. A solvent or composition is said to be “non-volatile” if it is not volatile.

A solvent or composition is said to be “non-aqueous” if there is no added water in the solvent or composition. That is to say, as used herein, a non-aqueous composition is one in which water has not been added as a component. For clarity, a solution or composition can be considered to be non-aqueous even if it contains water arising from a composition component, as long as no free water is added to the composition. Many of the solvents described herein are hydroscopic to a greater or lesser extent and such solvents may be part of a non-aqueous composition without regard to the water that is naturally absorbed by such materials.

The abbreviation “(w/w)” indicates that relative concentrations of components in a composition are presented on a “weight for weight” basis (i.e. percentages refer to a percentage of the total weight), rather than on the basis of volume or some other basis. In reference to a solvate or hydrate of ebastine, weight percentages should be weight corrected to account for mass pertaining to solvent/or hydrate molecules contained in the drug source.

The term “closed,” such as in regard to a “closed vial,” refers to a vial that is sealed against the significant loss of solvent or other materials from the vial by evaporation. For the Examples described herein, closed glass vials refer to non-reactive borosilicate glass vials closed with a polyethylene cone-lined phenolic cap and sealed with parafilm. The compositions within the glass vials were protected from light, either by using amber glass vials or by wrapping the vials with aluminum foil.

The term “active pharmaceutical ingredient” means “any substance or mixture of substances intended to be used in the manufacture of a drug product and that, when used in the production of a drug, becomes an active ingredient in the drug product. Such substances are intended to furnish pharmacological activity or other direct effect in the diagnosis, cure, mitigation, treatment or prevention of disease or to affect the structure and function of the body.” International Conference on Harmonization of Technical Requirements for Registration of Pharmaceuticals for Human Use, ICH Q7, Good Manufacturing Practice Guidance for Active Pharmaceutical Ingredients.

The term “pharmaceutically acceptable” in reference to an entity or ingredient is one that may be included in the composition provided herein and that causes no significant adverse toxicological effects in the patient at specified levels, or if levels are not specified, in levels known to be acceptable by those skilled in the art. All ingredients in the composition described herein are provided at levels that are pharmaceutically acceptable. For clarity, active pharmaceutical ingredients may cause one or more side effects and inclusion of the ingredients with a side effect profile that is acceptable from a regulatory perspective for such ingredients will be deemed to be “pharmaceutically acceptable” levels of those ingredients.

“Pharmaceutically acceptable salt” denotes a salt form of a drug or active pharmaceutical ingredient having at least one group suitable for salt formation that causes no significant adverse toxicological effects to the patient. Reference to an active pharmaceutical ingredient as provided herein is meant to encompass its pharmaceutically acceptable salts, as well as solvates and hydrates thereof. Pharmaceutically acceptable salts include salts prepared by reaction with an inorganic acid, an organic acid, a basic amino acid, or an acidic amino acid, depending upon the nature of the functional group(s) in the drug. Suitable pharmaceutically acceptable salts include acid addition salts which may, for example, be formed by mixing a solution of a basic drug with a solution of an acid capable of forming a pharmaceutically acceptable salt form of the basic drug, such as hydrochloric acid, iodic acid, fumaric acid, maleic acid, succinic acid, acetic acid, citric acid, tartaric acid, carbonic acid, phosphoric acid, sulfuric acid and the like. Typical anions for basic drugs, when in protonated form, include chloride, sulfate, bromide, mesylate, maleate, citrate, phosphate, and the like. Suitable pharmaceutically acceptable salt forms and methods for identifying such salts are found in, e.g., Handbook of Pharmaceutical Salts: Properties, Selection and Use, Weinheim/Zuerich:Wiley-VCH/VHCA, 2008; P. H. Stahl and C. G. Wermuth, Eds.

“Non-irritating” in reference to a topical formulation as provided herein refers to a formulation having an average score of less than 0.50 on the modified Draize scale for a test of three 6-week-old, male C57BL/6J mice. The modified Draize test is an acute erythema test carried out as follows. A C57BL/6J mouse is shaved in an application area, and the application area allowed to rest for approximately 24 hours and then rinsed with non-irritating soap. A test composition is applied evenly, without significant rubbing, to a 12 cm² area of the mouse's skin in a volume of 2.5 mg/cm². The sample is allowed to sit uncovered for 24 hours. After 24 hours, the application area is washed gently with lx phosphate buffered saline (1×PBS) and non-irritating soap to facilitate observation of the application area. The application area is then scored according to the following scale: 0=no evidence of erythema; 1=minimal erythema, barely perceptible; 2=definite erythema, readily visible, minimal edema or minimal popular response; 3=erythema and papules; 4=definite edema; 5=erythema, edema, and papules; 6=vesicular eruption; 7=strong reaction spreading beyond test site.

“Therapeutically effective amount” is used herein to mean the amount of a pharmaceutical preparation, or amount of an active pharmaceutical ingredient in the pharmaceutical preparation, that is needed to provide a desired level of active pharmaceutical ingredient in a target tissue or at a target site. The precise amount will depend upon numerous factors, e.g., the particular active pharmaceutical ingredient, the components and physical characteristics of the pharmaceutical preparation, intended patient population, patient considerations, and the like, and can readily be determined by one skilled in the art, based upon the information provided herein and available in the relevant literature.

Room temperature refers to a temperature in a range of about 20-25 degrees centigrade. In reference to a measurement or other feature requiring a precise indication of room temperature, room temperature is taken as 25 degrees centigrade.

The term “patient” refers to a human or other mammal suffering from or prone to a condition that can be prevented or treated by administration of a composition as provided herein.

“Optional” or “optionally” means that the subsequently described circumstance may or may not occur, so that the description includes instances where the circumstance occurs and instances where it does not.

In many cases, the patent application describes ranges of values. Such ranges shall be construed to include the endpoints of the range unless doing so would be inconsistent with the text or otherwise noted.

### Overview

The present disclosure overcomes one or more of the problems associated with a pharmaceutical composition comprising ebastine. Applicants have discovered a topical composition and related solvent system in which ebastine is delivered to the targeted mammal or human patient skin in quantities sufficient to treat a hair-loss related condition or disease, while avoiding large dosages associated with systemic delivery of oral ebastine compositions. The topical ebastine composition remains stable and sufficiently soluble, both in composition and when applied to the skin.

The present disclosure relates generally to a topical pharmaceutical composition and methods for preparing and using such a composition. Provided herein is a topical pharmaceutical composition, comprising ebastine dissolved in a solvent. In some embodiments, the solvent comprise a volatile solvent and a non-volatile solvent. In some embodiments, the solvent is non-aqueous.

Formulated for topical administration, the composition is advantageous in overcoming the problems of low bioavailability associated with oral ebastine's slow rate of dissolution in the aqueous medium of the stomach. A topical composition that delivers a drug primarily to the skin, rather than primarily systemically, ensures local therapeutic effects while avoiding large dosages required for bioavailable oral ebastine compositions.

In one or more embodiments, the volatile solvent is a monohydric aliphatic alcohol and the non-volatile solvent is an ester. Individually, as shown in Example 1 and Example 6, monohydric aliphatic alcohols and esters are both suitable solvents for ebastine. The monohydric aliphatic alcohol and ester-based combination solvent system is advantageous in overcoming the problems associated with solvent evaporation and drug insolubility. Relative to the individual components, the monohydric aliphatic alcohol and ester-based combination solvent system results in improved solubility of ebastine.

In one or more embodiments, the monohydric aliphatic alcohol is volatile, the ester is a non-volatile acid ester, and ebastine remains soluble in the composition when applied to skin. Volatile solvents or compositions typically evaporate readily at room temperature and atmospheric pressure. Examples of volatile solvents include isopropanol, ethanol, and t-butyl alcohol. Examples of non-volatile solvents include water, white petrolatum, and olive oil. More particularly, when the composition is applied to the skin, some of the solvent may be lost prior to skin penetration due to evaporation. This results in an increased concentration of ebastine in the composition on the skin surface, resulting in solid deposits of ebastine on the skin surface. Applicants have developed a composition in which ebastine remains soluble in the non-volatile acid ester even when the monohydric aliphatic alcohol concentration is reduced, such as through evaporation. In such embodiments, a favorable balance is achieved between the stabilizing and solubilizing effects of the non-aqueous co-solvent system on the composition (e.g., during storage), and the persistent solubility of the drug both in the composition and when applied to the skin, even upon evaporation of some or all of the monohydric aliphatic alcohol. Relative weight percentages of each of the volatile monohydric aliphatic alcohol, the non-volatile acid ester and ebastine in the composition, effective to achieve such a favorable balance are described herein.

Some embodiments of the composition beneficially further comprise water. Use of water alone as the solvent does not allow good solubility for ebastine. However, water may be beneficially combined with one or more other solvents to produce a composition in which ebastine is dissolved and the composition has other desirable characteristics. For example, certain non-aqueous solvents, such as monohydric aliphatic alcohols, or more particularly ethanol, are known to dry the skin and cause skin irritation. So, although ethanol alone is useful in solubilizing and stabilizing ebastine and in enabling effective penetration of ebastine, it causes skin drying and irritation. A composition that combines water and a monohydric aliphatic alcohol can advantageously avoid or minimize skin drying and irritation caused by high alcohol content and also maintain ebastine in solution. The relative weight percentages of each of ebastine, the alcohol, and water effective to achieve such a composition are described herein.

In some embodiments, the composition is used for the treatment of a hair-loss related condition or disease. An example of a hair-loss related condition or disease for which the composition may be used is androgenetic alopecia. Other examples of suitable indications include scarring alopecias and non scarring alopecias, including alopecia areata.

In some embodiments, the composition is used for the treatment of a dermatological disorder that benefits from antihistamine activity, such as ebastine. Examples of dermatological disorders for which the composition may be used are contact dermatitis, seborrheic dermatitis, or atopic dermatitis, including eczema.

### Ebastine Topical Compositions

As described above, the topical pharmaceutical composition comprises ebastine dissolved in a solvent. In some embodiments, the solvent comprises a volatile solvent and a non-volatile solvent. In some embodiments, the solvent is non-aqueous. Composition components and features will now be described in greater detail.

Ebastine includes ebastine and its corresponding pharmaceutically acceptable salt forms, as well as solvates and hydrates thereof. Ebastine is a potent and long-lasting second-generation antihistamine. It is preferred for use in the composition and methods of treatment for hair-loss related conditions or diseases provided herein because of its effectiveness, as ebastine or its active metabolite, carebastine, in decreasing PGD2 levels. This allows ebastine to act as a PGD2 inhibitor in cells lining the hair follicle (dermal papilla cells, hair follicle stem cells, root sheath cells) without a central side effect.

The amount of ebastine in the topical pharmaceutical composition typically ranges from about 0.01 percent to about 30 percent by weight, or from about 0.1 percent to about 20 percent by weight. Illustrative ranges are from about 0.1 percent to about 10 percent by weight or about 0.1 percent to about 5 percent by weight or 0.1 percent to 2.5 percent by weight. Additional illustrative ranges include from about 10 percent to about 20 percent by weight or 10 percent to 15 percent by weight, or about 20 percent to about 30 percent by weight or 20 percent to 25 percent by weight . For example, the topical formulation may comprise any one of the following weight percentages of ebastine: 0.1 percent, 0.2 percent, 0.3 percent, 0.4 percent, 0.5 percent, 0.6 percent, 0.7 percent, 0.8 percent, 0.9 percent, 1.0 percent, 1.1 percent, 1.2 percent, 1.3 percent, 1.4 percent, 1.5 percent, 1.6 percent, 1.7 percent, 1.8 percent, 1.9 percent, 2.0 percent, 2.1 percent, 2.2 percent, 2.3 percent, 2.4 percent, 2.5 percent and so forth. In a preferred embodiment, the amount of ebastine in the topical pharmaceutical composition is 2.0 percent by weight.

The topical ebastine composition may comprise a volatile solvent, preferably a monohydric aliphatic alcohol. A monohydric aliphatic alcohol is a preferred volatile solvent for use in the composition and methods provided herein due to its permeation and penetration enhancing activities. Such alcohols also have good ebastine solubility. Preferably, the monohydric aliphatic alcohol is a primary alcohol such as ethyl alcohol, propyl alcohol or butyl alcohol. One particularly preferred monohydric aliphatic alcohol is ethanol, as described in more detail in Examples 1 and 2.

Many solvents, particularly monohydric aliphatic alcohols and more particularly ethanol, can provide a stable solvent for ebastine. However, since ethanol is a volatile solvent, much of the solvent concentration is reduced through evaporation when the composition is resident on the skin. Following topical application, this evaporation combined with penetration of ethanol into the skin can result in deposition of ebastine precipitates on the skin surface or in the upper layers of the skin. Precipitation is not desirable as it limits the amount of ebastine that can be absorbed by the skin from application of the composition.

Some embodiments of the topical pharmaceutical composition beneficially further comprise a non-volatile solvent. This non-volatile solvent can be an ester containing one or more alkoxy groups. Preferred esters are diethyl sebacate, ethyl acetate, and dimethyl succinate as described in the results reported in Example 6.

Non-volatile acid esters can be included as a component of the solvent system in order to address the shortcomings of monohydric aliphatic alcohol evaporation leading to precipitation on the skin surface. For example, in a composition comprised of ebastine, ethanol, and diethyl sebacate, ethanol is more volatile than diethyl sebacate, such that even if substantially all ethanol evaporates from the composition, essentially all of the ester remains in the composition. Ebastine is soluble in the ester. For example, approximately 120.6 mg ebastine is soluble in 1 gram of diethyl sebacate. This means that in the foregoing example, the ebastine will desirably remain in solution (i.e., in a dissolved state) at skin temperature for many desirable concentrations of ebastine, such as concentrations of ebastine of 0.1% w/w to 10.0% w/w. This is advantageous as it provides for the solubility of ebastine in composition before it is applied to the skin and during the period when the composition is resident on the skin, to achieve skin penetration of ebastine over a period of time greater than achieved from a composition in which the solvent is solely comprised of a volatile solvent.

Exemplary compositions as provided herein comprise a greater percent by weight of the monohydric aliphatic alcohol in comparison to the ester. For example, advantageous compositions as described herein may comprise from about 50 percent (w/w) to about 95 percent (w/w) monohydric aliphatic alcohol, from about 5 percent (w/w) to about 40 percent (w/w) ester, and from about 0.01 percent (w/w) to about 5 percent (w/w) ebastine. Some preferred compositions as described herein may comprise from about 60 percent (w/w) to about 85 percent (w/w) monohydric aliphatic alcohol, from about 15 percent (w/w) to about 35 percent (w/w) ester, and from about 0.1 percent (w/w) to about 3 percent (w/w) ebastine.

Illustrative liquid compositions may contain, for example, any one or more of the following weight-weight (w/w) percentages of monohydric aliphatic alcohol, including ranges between each of the following values: 5 percent, 10 percent, 15 percent, 20 percent, 25 percent 30 percent, 35 percent, 40 percent, 45 percent, 50 percent, 55 percent, 60 percent, 65 percent, 70 percent, 75 percent, 80 percent, 85 percent, 90 percent and 95 percent alcohol (w/w), where preferably, the weight percent alcohol is greater than the weight percent ester. Further representative ranges for the alcohol component, which may be combined with w/w amounts or ranges for ebastine and other formulation components as provided herein are from: 50-55 percent w/w, 50-60 percent w/w, 50-65 percent w/w, 50-70 percent w/w, 50-75 percent w/w, 50-80 percent w/w, 50-85 percent w/w, 50-90 percent w/w, 50-95 percent w/w, 60-65 percent w/w, 60-70 percent w/w, 60-75 percent w/w, 60-80 percent w/w, 60-85 percent w/w, 60-90 percent w/w, 60-95 percent w/w, 65-70 percent w/w, 65-75 percent w/w, 65-80 percent w/w, 65-85 percent w/w, 65-90 percent w/w, 65-95 percent w/w, 70-75 percent w/w, 70-80 percent w/w, 70-85 percent w/w, 70-90 percent w/w, 70-95 percent w/w, 75-80 percent w/w, 75-85 percent w/w, 75-90 percent w/w, 75-95 percent w/w, 80-85 percent w/w, 80-90 percent w/w, 85-95 percent w/w, and 90-95 percent w/w.

Representative amounts of an ester component, include, any one or more of the following: 5 percent, 10 percent, 15 percent, 20 percent, 25 percent 30 percent, 35 percent, 40 percent, 45 percent, 50 percent, 55 percent, 60 percent, 65 percent, 70 percent, 75 percent, 80 percent, 85 percent, 90 percent, and 95 percent (w/w), where preferably, the weight percent alcohol is greater than the weight percent ester. Further representative ranges for the ester component, which may be combined with w/w amounts or ranges for ebastine and other formulation components as provided herein are from: 5-10 percent w/w, 5-15 percent w/w, 5-20 percent w/w, 5-25 percent w/w, 5-30 percent w/w, 5-35 percent w/w, 5-40 percent w/w, 10-15 percent w/w, 10-20 percent w/w, 10-25 percent w/w, 10-30 percent w/w, 10-35 percent w/w, 10-40 percent w/w, 15-20 percent w/w, 15-25 percent w/w, 15-30 percent w/w, 15-35 percent w/w, 15-40 percent w/w, 20-25 percent w/w, 20-30 percent w/w, 20-35 percent w/w, 20-40 percent w/w, 25-30 percent w/w, 25-35 percent w/w, 20-40 percent w/w, 25-30 percent w/w, 25-35 percent w/w, 25-40 percent w/w, 30-35 percent w/w, 30-40 percent w/w, and 35-40 percent w/w.

The ratio between the monohydric aliphatic alcohol and the ester can be in a range of 1:1 to 1:99 or 1:1 to 99:1 by weight. As set forth above, the composition will preferably comprise a greater percent by weight of the monohydric aliphatic alcohol in comparison to the ester. Exemplary w/w ratios of alcohol to ester include, for example, about 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 65:1, 70:1, 75:1, 80:1, 85:1, 90:1, 95:1, and 99:1. The composition may comprise a w/w ratio between the monohydric aliphatic alcohol and the ester between about 1:1 to 20:1, or from about 1:1 to about 10:1, or from about 5:1 to about 25:1, or from about 5:1 to about 15:1, or from about 2:1 to about 19:1, or from about 2:1 to about 9:1. The composition may comprise a greater percent by weight of the ester in comparison to the monohydric aliphatic alcohol. Exemplary w/w ratios of alcohol to ester include, for example, about 1:2, 1:3, 1:4, 1:5, 1:6, 1:7, 1:8, 1:9, 1:10, 1:15, 1:20, 1:25, 1:30, 1:35, 1:40, 1:45, 1:50, 1:60, 1:65, 1:70, 1:75, 1:80, 1:85, 1:90, 1:95, and 1:99. The composition may comprise a w/w ratio between the monohydric aliphatic alcohol and the ester between about 1:1 to 1:20, or from about 1:1 to about 1:10, or from about 1:5 to about 1:25, or from about 1:5 to about 1:15, or from about 1:2 to about 1:19, or from about 1:2 to about 1:9.

A further component of the topical composition (i.e., forming part of its solvent system) may be a polyol containing two or more hydroxyl groups, and possessing from 3-8 carbon atoms. Typically, the polyol is an aliphatic compound; polyols for use in the instant composition include diols such as propylene glycol (PG, propane-1,2-diol), hexylene glycol (2-methylpentane-2,4-diol), 1,3-butylene glycol (1,3-butane diol), and dipropylene glycol, triols such as glycerol and trimethylolpropane, and higher alcohols (meaning containing more than 3 hydroxyl groups) such as sorbitol and pentaerythritol. Preferred polyols are C3-C8 diols and triols. The diol or triol will typically have a molecular weight less than about 250, or even less than about 200. In some instances, the polyol will have a molecular weight less than about 125. The polyol, may, in some instances, be hygroscopic, such as in the case of propylene glycol. In some embodiments, the polyol is a triol other than glycerol or glycerin.

Polyols are included as an additional component of the solvent system described above for their penetration enhancing activities (i.e., smoothing, hydrating, and humectant effects). Although ebastine exhibits limited solubility in polyols, particularly propylene glycol, the combination of ebastine, a monohydric aliphatic alcohol, an ester, and a polyol maximizes solubility in the composition and maximizes skin penetration of ebastine when the composition is applied to the skin.

Some embodiments of the topical pharmaceutical composition beneficially further comprise water. As seen in Example 3, ebastine is insoluble in water for pH values of 5 and above. pH values below 5 are typically irritating to the skin and so less desirable. Therefore, water is typically not included herein as the sole or primary component of the solvent system. Instead, it is more typically used herein as a supplemental component of the composition for the purpose of overcoming the other formulation shortcomings, such as skin drying and irritation associated with high alcohol content. More specifically, while alcohol is useful in solubilizing and stabilizing ebastine and enabling effective penetration of ebastine, including water and decreasing alcohol content is advantageous in overcoming skin drying and irritation caused by high alcohol content. The ratio between the non-aqueous solvent and water can be in a range of 1:1 to 99:1 by weight. As set forth above, the composition will preferably comprise a greater percent by weight of the non-aqueous solvent in comparison to the water. Exemplary w/w ratios of non-aqeous solvent to water include, for example, about 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 65:1, 70:1, 75:1, 80:1, 85:1, 90:1, 95:1, and 99:1. The composition may comprise a w/w ratio of the non-aqueous solvent and water between about 1:1 to 20:1, or from about 1:1 to about 10:1, or from about 5:1 to about 25:1, or from about 5:1 to about 15:1, or from about 2:1 to about 2:1 to about 19:1, or from about 2:1 to about 9:1. In a preferred embodiment, the ratio between the non-aqueous solvent and water is 9:1 by weight, as described in the results reported in Example 4. In some preferred embodiments, the non-aqueous solvent and water comprise at least 90%, 95%, or 98% of the composition.

The topical composition may be in a number of different forms, including, for example, a solution, liquid, spray, foam, lotion, gel and the like. Preferably, the composition is a liquid, has good stability, adheres to the skin, and has a smooth feel. For additional information regarding suitable formulations, see, for example, “Remington: The Science and Practice of Pharmacology,” 22nd edition, (Pharmaceutical Press, 2013).

### Methods for Preparation, Methods of Use and Kits

A wide variety of methods may be used for preparing the composition described herein. Broadly speaking, the composition may be prepared by combining together the components of the composition, as described herein, at a temperature and for a time sufficient to provide a pharmaceutically effective and desirable composition. The term “combining together”, as used herein, means that all of the components of the composition are combined and mixed together at about the same time, or that various components are combined in one or more sequences or orders of addition to provide the desired product. The composition can be prepared on a weight/weight (w/w) or a weight/volume (w/v) basis. The composition preferably are readily spreadable, e.g., on a surface of the skin, and preferably will not be runny.

The composition may be prepared by, e.g., admixture of the ingredients typically through the use of vigorous agitation such as high shear mixing. Mixing can also be accomplished by any suitable method using any suitable manual or automated means. Optional additional steps include those which result in the addition of one or more of the optional auxiliary ingredients as set forth above. Methods for preparing a pharmaceutical formulation are well known in the art and are described, for example, in Handbook of Pharmaceutical Formulations: Liquid Products, Vol 3, S. Niazi., CRC Press, 2004.

A wide variety of methods may be used for administering the composition described herein. One such method is for the treatment of hair-loss related conditions or diseases and comprises applying the composition described herein to a targeted mammal or human patient surface at least once daily for a period of at least one month. Another such method is for the treatment of dermatological disorders that benefit from antihistamine activity and comprises applying the composition described herein to a targeted mammal or human patient surface at least once daily for a period of at least one week.

The composition provided herein is useful for treatment of hair-loss related conditions or diseases, such as scarring alopecia and non scarring alopecia, including androgenetic alopecia and alopecia areata. In a preferred embodiment, the for treatment of hair-loss related conditions or diseases is treatment of androgenetic alopecia, affecting either men or women.

The composition provided herein is useful for treating dermatological disorders that benefit from antihistamine activity such as ebastine. The composition provided herein may be used, for treating conditions such as, for example, contact dermatitis, seborrheic dermatitis, or atopic dermatitis, including eczema.

Skin surfaces of all types, for example the scalp, facial skin, chest skin, arms, legs, pubic region, etc., may be targeted for application within the composition described herein. The human scalp is the preferred targeted surface for use in the methods of administration provided herein as it is the most visible as well as the most commonly affected area from hair-loss related conditions and diseases.

Typically, the composition is applied in a conventional amount from once to several times weekly or daily on the targeted areas of the skin, until the treated condition or disease has noticeably subsided. A conventional amount is an amount that is sufficient to spread, e.g., thinly spread, over the affected area. For example, the composition may be applied topically at least once daily for a period of at least 1 month. The frequency and number of applications and the course of treatment will vary based on condition severity, patient considerations, etc. Thus, the composition may, in certain instances by applied one daily, twice daily, three times daily, once every other day, from one to three times weekly, from one to four times weekly, every three days, etc.

The composition may be topically applied directly to the targeted surface, for example, using the fingertips, a sponge applicator, a cotton applicator, by spraying, aerosolization, or any other suitable method.

In one or more embodiments, the efficacy of treatment is assessed by a visual examination of the affected area. In some cases, prophylactic treatment may be continued even if the condition has visibly diminished or disappeared, as a preventative measure.

According to one or more embodiments, there is also provided a kit for the delivery of a topical pharmaceutical composition comprising a container closure, a topical ebastine composition as described herein, and instructions for use. In some embodiments, the container closure comprises a nozzle capable of releasing the topical pharmaceutical composition. The instructions for use may include instructions for application of the topical pharmaceutical composition described herein. The instructions may comprise instructions to apply the composition to the mammal or human skin surface, such as the scalp, once daily for a period of at least one week or at least one month or until the treated condition or disease has noticeably subsided.

### EXAMPLES

The following examples provide those of ordinary skill in the art with a description of embodiments of the composition, their characteristics, and methods of their preparation and use.

### Example 1

### Solubility of Ebastine in Ethanol

A study was performed to assess the solubility of the composition containing ebastine. Composition EBA-E was formed by dissolving ebastine (AvaChem Scientific, San Antonio, Tex.) in ethanol (Spectrum Chemical, Gardena, Calif.) at room temperature and atmospheric pressure.

To evaluate solubility, a measured amount of ebastine was placed in a 20 mL glass scintillation vial. Measured quantities of ethanol were added to the ebastine in the glass vial, the lid was placed on the vial, and the vial was mixed using a magnetic stirrer. Addition of the solvent was repeated until the test material was completely dissolved, resulting in a visually clear solution.

The resulting solubility concentration, the highest concentration that dissolved ebastine in the solvent, was 28.17 (mg/g) at room temperature. This result demonstrates that ethanol is a solvent.

### Example 2

### Stability of Ebastine in Ethanol Compositions

The effect of component contributions to drug potency stability for an illustrative mixture of ebastine and ethanol was assessed using the composition described in Example 1.

The degradation and stability of the ebastine composition was measured at 1-day, 1-week, 2-week, 3-week, and 2-month time points following storage in a 40° C. oven within sealed clear glass vials. Table 1 below presents the concentration of ebastine for the composition described in Example 1 normalized by all peak areas at each of the above-mentioned time points.

TABLE 1
Concentrations of Ebastine Relative to Initial Concentration Measurement

| Sample | | | | | | Concentration |
|---|---|---|---|---|---|---|
| Designation | | | | | | Change (difference |
| (See Example 1) | Day 1 | Week 1 | Week 2 | Week 3 | 2 months | in concentrations) |
| EBA-E | 99.99% | 99.75% | 103.03% | 102.09% | 101.38% | +1.39% |

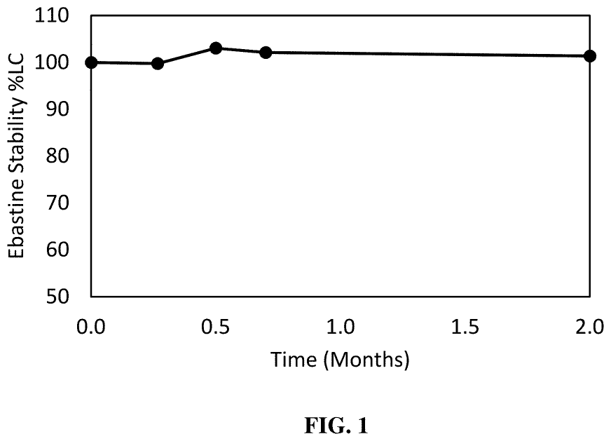

Results in Table 1 and in FIG. 1 demonstrate that ebastine in ethanol is stable over the tested period. Small changes in ebastine measurements that result in measurements that exceed 100% are understood to be based on measurement error.

### Example 3

### Solubility of Ebastine in Water

A study was performed to assess the solubility of the composition containing ebastine. Composition EBA-W was formed by dissolving ebastine (AvaChem Scientific, San Antonio, Tex.) in deionized (DI) water (LabChem, Inc., Zelienople, Pa.). Solubility of ebastine in water was assessed at pH values of the composition in the range of 3-7 at room temperature.

For each pH measurement, to evaluate solubility, a measured amount of ebastine was placed in a 20 mL glass scintillation vial. Measured quantities of water were added to the ebastine in the glass vial, the lid was placed on the vial, and the vial was mixed using a magnetic stirrer. The pH of each suspension was determined using a pH meter (Orion Star A111, Thermo Scientific, Waltham, Mass.). Following, the pH of each suspension was adjusted with acid 1N HCl to the target pH. A 2 milliliter (mL) sample of the liquid portion of the mixture (supernatant) was removed from the top of each vial and spun in a micro-centrifuge (Eppendorf Minispin, Hamburg Germany) at 12,000 rpm for 2 minutes. A 10 microliter (μL) portion of each of the resulting mixtures was used in an HPLC method for assessment of ebastine concentration as described in the next paragraph.

A 10 microliter (μL) sample was injected into a high-performance liquid chromatography machine (HPLC) (Agilent, Santa Clara, Calif.). For ebastine analysis, the HPLC column (Phenomenex, Inc. Torrance, Calif.) was a C-18 column 100×4.6 mm with a particle size of 5 micrometers (micron). The HPLC system also used a guard column (Phenomenex, Inc.) and a mobile phase consisting of a base solvent of 70 percent (v/v) methanol (Spectrum Chemicals, Gardena, Calif.), and 30 percent (v/v) 40 mM potassium phosphate buffer, pH 4.0 (Spectrum Chemicals, Gardena, Calif.). The HPLC flow rate was 1 mL per minute with a column temperature of 40 degrees centigrade, a detection wavelength of 262 nanometers (nm), and a runtime of at least 15 minutes.

The resulting concentrations describe the ebastine concentration that was dissolved in water after pH adjustments, at room temperature, as described in this example above.

TABLE 2
Ebastine Concentrations in Water at Selected pH Values

| pH | 3.04 | 5.06 | 6.91 |
|---|---|---|---|
| Ebastine Solubility | 1.4 (mg/g) | 0 (mg/g) | 0 (mg/g) |

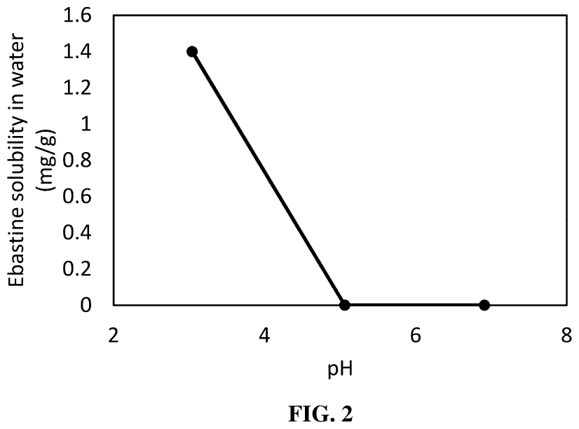

The resulting concentrations are listed in Table 2 and shown in FIG. 2. The results demonstrate that ebastine is insoluble in water when the pH of the resulting composition is in the range of 5 to 7 at room temperature and only slightly soluble when that pH range is below 5.0, such as 3.0 to 5.0.

### Example 4

### Solubility of Ebastine in Ethanol:Water

A study was performed to assess the solubility of the composition containing ebastine. Composition EBA-EW was formed by dissolving ebastine (AvaChem Scientific, San Antonio, Tex.) in a mixture of ethanol (Spectrum Chemical, Gardena, Calif.) and DI water (LabChem, Inc. Zelienople, Pa.) in a ratio of 9:1. Solubility at room temperature of ebastine in this 9:1 ethanol:water mixture was assessed for compositions with pH values in the range from 2 to 10 using the same steps described in Example 3.

TABLE 3
Ebastine Concentrations in 90:10 Ethanol:Water

| pH | 2.3 | 6.16 | 7.76 | 9.90 |
|---|---|---|---|---|
| Ebastine | 30.38 (mg/g) | 33.35 (mg/g) | 16.58 (mg/g) | 15.3 (mg/g) |
| Solubility | | | | |

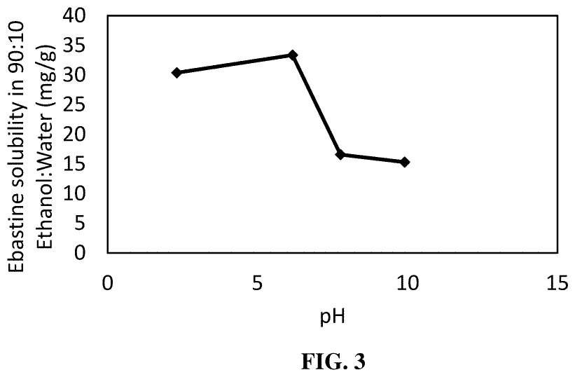

The resulting concentrations are listed in Table 3 above and in FIG. 3. The results demonstrate that ebastine is soluble in 9:1 ethanol:water in the pH range of from 2 to 6 and is at least sparingly soluble for pH values above 6, such as the pH range of 7 to 10. Thus, ebastine was demonstrated to be much more soluble in a mixture of ethanol and water than in water alone. Notably, the solubility of ebastine in a composition with a pH value of less than about 7, such as with a pH value of 2.3 to 6.2, and comprising a mixture of ethanol and water beneficially exceeded the solubility of ebastine in ethanol alone (approximately 22 mg/g) or in water alone (less than 2 mg/g for useful pH ranges; see, e.g., Example 2).

### Example 5

### Stability of Ebastine in Ethanol:Water

The effect of component contributions to drug potency stability for an illustrative composition comprising ebastine dissolved in a 9:1 of ethanol and water was assessed for composition pH values in the range of 2 to 6.

The degradation and stability of the ebastine composition was measured at 1-day, 1-week, 1-month, 2-month, and 3-month time points following storage in a 40° C. oven within sealed clear glass vials. Table 4 below presents the concentration of ebastine normalized by all peak areas at each of the listed time points.

TABLE 4
Concentrations of Ebastine in Ethanol: Water Relative to Initial Concentration Measurement

| pH | | | | | | Concentration Change |
|---|---|---|---|---|---|---|
| Values | Day 1 | Week 1 | Month 1 | Month 2 | Month 3 | (difference in concentration) |
| pH 2 | 99.96% | 99.99% | 97.23% | 94.65% | 90.41% | −9.55% |
| pH 3 | 99.96% | 99.98% | 99.82% | 99.88% | 99.90% | −0.06% |
| pH 4 | 99.96% | 99.99% | 99.78% | 99.90% | 99.82% | −0.14% |
| pH 5 | 99.97% | 99.98% | 99.81% | 99.90% | 99.81% | −0.16% |
| pH 6 | 99.96% | 99.98% | 99.78% | 99.88% | 99.79% | −0.17% |

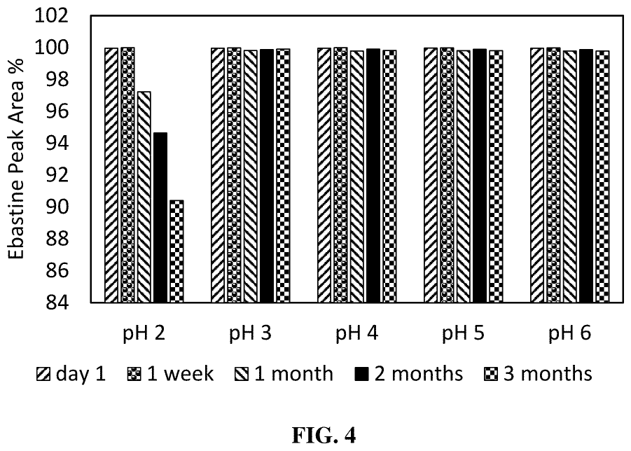

Results in Table 4 and in FIG. 4 demonstrate that ebastine is stable over the tested period when dissolved in a 9:1 mixture of ethanolwater in compositions with pH in the range from 3 to 6. Ebastine is not stable in 9:1 mixtures of ethanol and water for compositions with a pH value of 2 after a period of 1-week at 40° C.

### Example 6

### Solubility of Ebastine in Ester Solvents

A study was performed to assess the solubility of the composition containing ebastine. Compositions described in Table 5 below were formed by dissolving ebastine (AvaChem Scientific, San Antonio, Tex.) in various esters. To evaluate solubility, a measured amount of ebastine was placed in a 20 mL glass scintillation vial. Measured quantities of ester solvents were added to the ebastine in the glass vial, the lid was placed on the vial, and the vial was mixed using a magnetic stirrer. Addition of the solvent was repeated until the test material was completely dissolved, resulting in a visually clear solution.

The resulting ebastine concentrations are listed in Table 5 below. The results demonstrate several esters which are compatible solvents for ebastine, at room temperature.

TABLE 5
Ebastine Concentrations in Individual Ester Solvent Systems

| | EBASTINE SOLUBILITY |
|---|---|
| ESTER SOLVENT | AT ROOM TEMP (mg/g) |
| Methylpyrrolidone | >23.1 |
| Benzyl Benzoate | >45.0 |
| Diethylene Glycol | 33.9 |
| Monoethyl Ether | |
| Diethyl Sebacate | 120.6 |
| Isopropyl Myristate | 21.8 |
| Diisopropyl Adipate | 28.7 |
| Triacetin | <1.1 |
| Ethyl Acetate | 245.2 |
| Dimethyl Succinate | 0.47 |

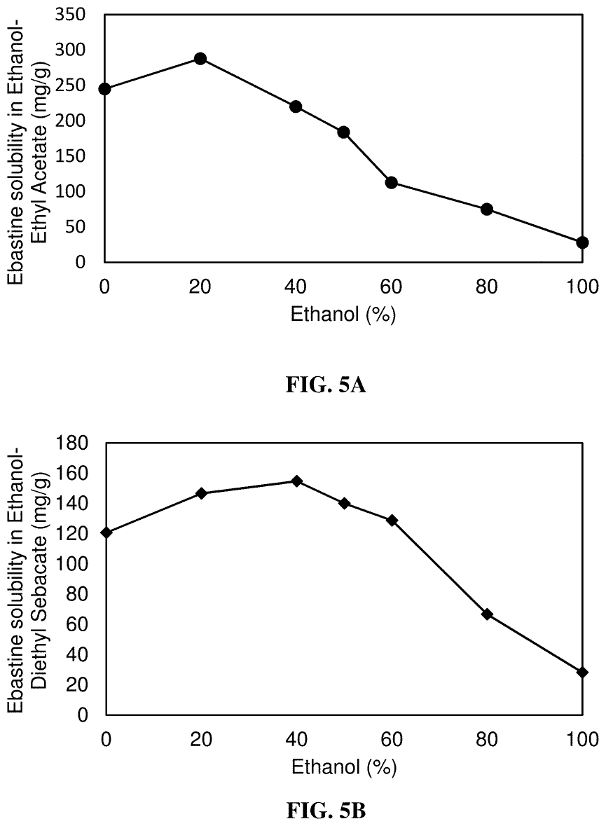

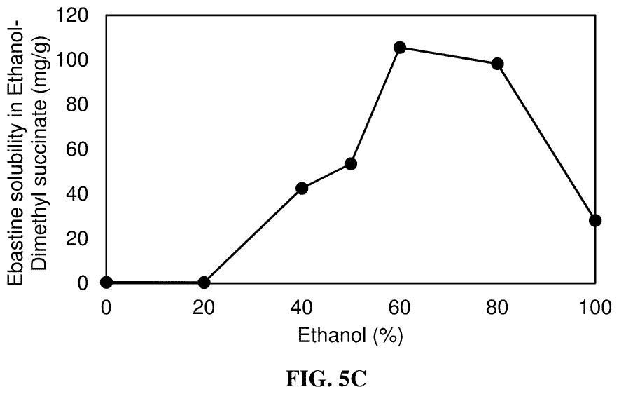

The solubility of ebastine was significantly improved in many cases by selected binary mixtures of ethanol and an ester, as shown in Table 6 below, relative to the solubility of ebastine in ethanol or the selected ester alone. As shown in Table 6 and in FIG. 5A ebastine solubility in compositions comprising 1% to 30% ethanol and 70% to 99% ethyl acetate is higher than ebastine solubility in either ethanol or ethyl acetate alone. As shown in Table 6 and in FIG. 5B ebastine solubility in compositions comprising 1% to 60% ethanol and 40% to 99% diethyl sebacate is higher than ebastine solubility in either ethanol or diethyl sebacate alone. As shown in Table 6 and in FIG. 5C ebastine solubility in compositions comprising 40% to 99% ethanol and 1% to 60% dimethyl succinate is higher than ebastine solubility in either ethanol or diethyl sebacate alone. Thus, the ebastine in selected ranges of binary mixtures was higher than the solubility of ebastine in the individual components of the solvent mixture. Even small amounts of esters can have large effects. For example, the solubility in the mixture of 80% ethanol and 20% dimethyl succinate is 98.3 mg/g compared to only 28.17 mg/g in ethanol alone and 0.47 in dimethyl succinate alone.

TABLE 6
Solubility of Ebastine in Binary Mixtures of Solvent Components

| | MIXTURE | EBASTINE |
|---|---|---|
| | RATIO | SOLUBILITY |
| SOLVENT COMPONENTS | (W/W) | (mg/g) |
| ethanol and ethyl acetate | 80:20 | 75.14 |
| ethanol and ethyl acetate | 60:40 | 112.83 |
| ethanol and ethyl acetate | 50:50 | 183.93 |
| ethanol and ethyl acetate | 40:60 | 219.97 |
| ethanol and ethyl acetate | 20:80 | 287.79 |
| ethanol and diethyl sebacate | 80:20 | 66.63 |
| ethanol and diethyl sebacate | 60:40 | 128.79 |
| ethanol and diethyl sebacate | 50:50 | 139.94 |
| ethanol and diethyl sebacate | 40:60 | 154.69 |
| ethanol and diethyl sebacate | 20:80 | 146.57 |
| ethanol and dimethyl succinate | 80:20 | 98.30 |
| ethanol and dimethyl succinate | 60:40 | 105.60 |
| ethanol and dimethyl succinate | 50:50 | 53.50 |
| ethanol and dimethyl succinate | 40:60 | 42.50 |
| ethanol and dimethyl succinate | 20:80 | 0.37 |

The combination of ethanol with an ester (e.g. ethyl acetate, diethyl sebacate, dimethyl succinate) resulted in mixtures which significantly improved the solubility of ebastine. Thus, the mixtures comprising ethanol and an ester were shown to form a solvent with beneficial and unexpected characteristics relative to the individual components.

### Example 7

### Exemplary Compositions

Table 7 provides illustrative topical compositions. The compositions were prepared by first mixing the components together (e.g., ethanol, propylene glycol, dimethyl succinate (Sigma-Aldrich, St. Louis, Mo.) or dimethyl sebacate (Spectrum Chemical, Gardena, Calif.)), then the ebastine drug was added to the solvent system with a thickener (e.g., hydroxypropyl cellulose HF).

TABLE 7
Exemplary Compositions (w/w)

| Formulation Number | EBA-A.1 | EBA-A.2 | EBA-A.3 | EBA-A.4 |
|---|---|---|---|---|
| Ebastine | 2.00% | 2.00% | 2.00% | 2.00% |
| Klucel HF | 0.60% | 0.60% | 0.60% | 0.60% |
| Ethanol, anhydrous | 61.25% | 61.25% | 77.40% | 77.40% |
| Propylene Glycol | 16.15% | 16.15% | — | — |
| Dimethyl succinate | 20.00% | — | 20.00% | — |
| Diethyl Sebacate | — | 20.00% | — | 20.00% |

### Example 8

### Stability of Exemplary Compositions

The effect of component contributions to drug potency stability was assessed for the compositions described in Table 7.

The degradation and stability of the ebastine composition was measured at 1-day, 1-week, 4-week, and 8-week time points following storage in a 40° C. oven within sealed clear glass vials. Table 8 below presents the concentration of ebastine normalized by all peak areas at each of the listed time points.

TABLE 8
Concentrations of Ebastine in Exemplary Compositions
Relative to Initial Concentration Measurement

| | | | | | Concentration |
|---|---|---|---|---|---|
| | | | | | Change |
| Sample | | | | | (difference in |
| Designation | Day 1 | Week 1 | Week 4 | Week 8 | concentration) |
| EBA-A.1 | 99.55% | 99.52% | 99.32% | 99.44% | −0.11% |
| EBA-A.2 | 99.32% | 99.81% | 99.74% | 99.53% | +0.21% |
| EBA-A.3 | 99.11% | 99.57% | 99.37% | 99.37% | +0.26% |
| EBA-A.4 | 99.32% | 99.81% | 99.68% | 99.50% | +0.18% |

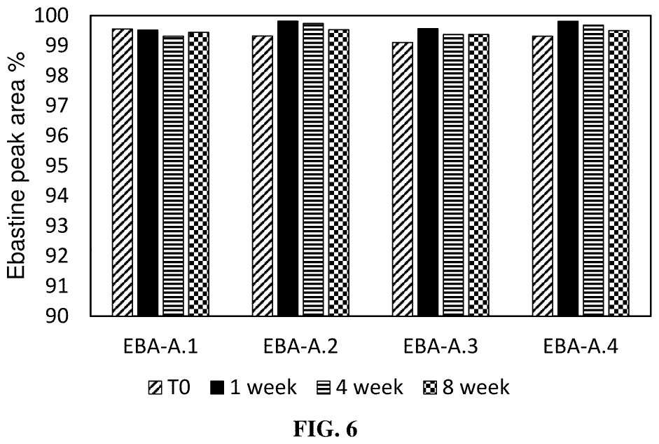

Results in Table 8 and in FIG. 6 demonstrate that ebastine is stable in exemplary compositions described in Table 7 over the tested period. Small changes in ebastine measurements that result in measurements that exceed 100% are understood to be based on measurement error.

### Example 9

### In Vivo Mouse Model Study

An in vivo experiment in mice was conducted to determine whether ebastine penetrates into the skin in a sufficient amount to achieve a desired therapeutic effect when administered from compositions comprising a monohydric aliphatic alcohol and a polyol. Penetration into the skin was assessed in a sample size of three male mice. The test composition was a mixture consisting of ebastine, ethanol, and propylene glycol.

A solvent mixture of ethanol (Spectrum Chemicals, Gardena, Calif.) and propylene glycol (Spectrum Chemicals, Gardena Calif.) was prepared by mixing the solvents in the proportions listed in Table 9. 2% (w/w) ebastine (AvaChem Scientific, San Antonio, Tex.) was added to the solvent mixture.

TABLE 9
Components for Composition Assessed
in Skin Penetration Experiment

| Component | % w/w |
|---|---|
| Ethanol | 73.5% |
| Propylene glycol | 24.5% |
| Ebastine | 2% |

After being acclimated for seven days, a total of six 6-week-old, male C57BL/6J mice were randomly assigned to two experimental groups: an ebastine treatment group (3 mice) and a non-treatment group (3 mice).

One day before first application of the ebastine composition (Day 1), the mice were anesthetized with 2% isoflurane and an approximately 12 cm² (4×3cm) patch towards the posterior dorsal end of each mouse was shaved with electric clippers. The skin surface of the shaved patch was wiped clean with phosphate buffer saline (1×PBS). Mice were left to acclimate overnight.

Prior to each daily dosing, the shaved sites were cleaned with a waterless shampoo and 1×PBS. For five weeks, the test composition was applied daily at a dose of 2.5 mg/cm² using a positive displacement pipette and carefully spread in the 12 cm² test area using a clean metal spatula.

For the mice in the treatment group, both skin concentrations and blood plasma levels of ebastine were assessed after the 5-week treatment period with the 2% ebastine composition.

### Hair Growth Length

At the end of the 5-week study period, 10 hairs from each site were plucked from the upper-middle, middle, and lower-middle patch area of each of the mice. In addition, 10 hairs were plucked from outside the patch area for each of the mice in the nontreatment group. The length of each of the plucked hairs was measured with a microcaliper and the resulting values were averaged.

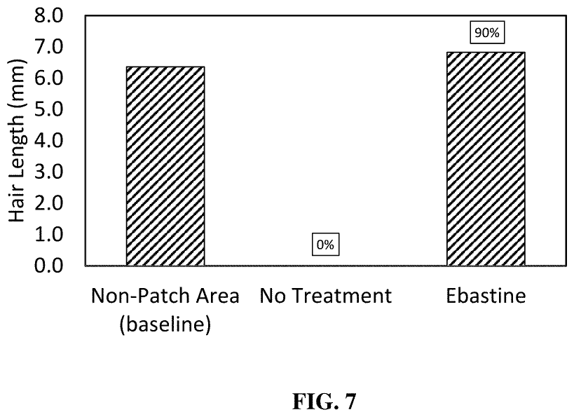

These average length values are presented in FIG. 7. Results indicate that mice treated with ebastine present increased hair length when compared with the baseline measurement (i.e., hair length of non-patch area of non-treated mice). Increased hair length is beneficial for treatments associated with a hair-loss related condition or disease because it helps to increase the volume of hair in such patients. The reported results thus suggest topical ebastine compositions can beneficially increase hair length when applied to mammal skin containing hair follicles and that such results indicate that topical ebastine compositions as described herein are useful in the treatment of patients suffering from a hair-loss related condition or disease.

Percentage of Patch Covered with Hair

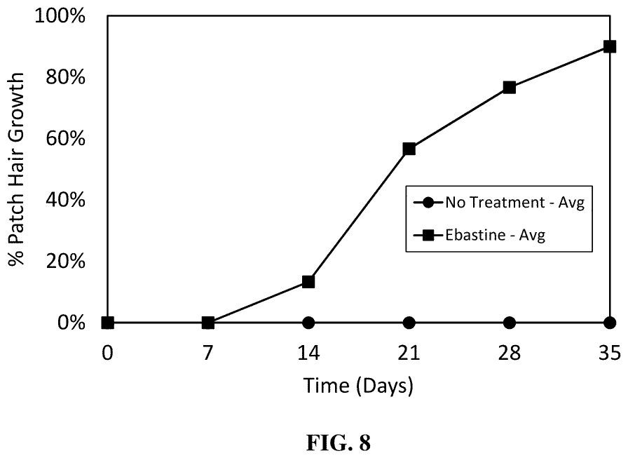

The percentage of the shaved patch that exhibited hair growth was measured weekly for each of the mice. The average values for mice in the ebastine treatment group and for mice in the nontreatment group are presented in FIG. 8. These data show that at study end (i.e., after 5 weeks), the total average patch coverage reached 90% of the total patch area for mice in the ebastine treatment group, while the nontreatment group presented no significant hair growth (i.e., total average patch coverage for the nontreatment group was 0% of the total patch area). The results demonstrate that topically applied ebastine compositions can effectively stimulate hair growth and that topical application of ebastine compositions, such as those described herein, provides a treatment for a hair-loss related condition or disease in mammals.

### Anagen Onset

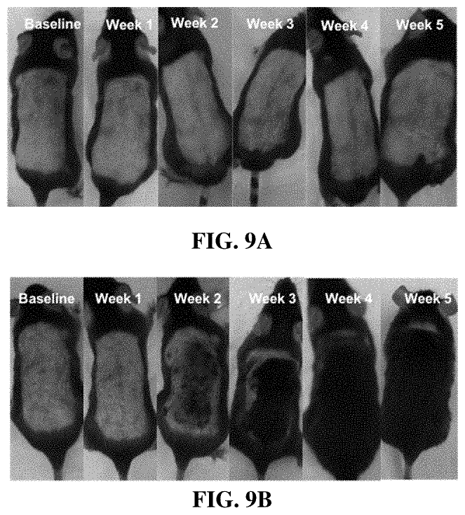

Mouse models are frequently used for studying the human hair cycle because they have synchronized hair growth cycles, which allows more conclusive data regarding hair cycle than studies on human subjects. In both human and mice, the hair stage cycle consists of three phases—anagen (growth phase), catagen (transition phase), and telogen (rest phase). In the human hair cycle, catagen phase is 7-14 days, and telogen phase is 2-4 months. In mice of the age under study, catagen phase is approximately 1 week, and telogen phase is approximately 5 weeks. The hair growth cyclicity through the anagen, catagen, and telogen phases can be used as a treatment assessment method for the hair growth promoting agent (i.e., ebastine). In the study (as described supra), mice were shaved while in the telogen phase. Shaving stimulated hair growth by inducing the transition from rest (telogen phase) to growth (anagen phase). The shaved patch area for each mouse was observed daily for anagen onset, i.e., the first day of the anagen phase of the hair cycle. In black-haired mice, new black hair growth under the skin surface is easily observed in the pink unpigmented skin. Therefore, anagen onset was recorded and determined to be the first day the mouse exhibits visible darkness through the skin that subsequently progresses to visible black hair. In mice in the ebastine treatment group, significant anagen induction was obtained on average after 10 days of treatment. (FIG. 9B). Non-treated mice did not show significant anagen induction until Day 35 (5.0 weeks). (FIG. 9A). These results demonstrate that ebastine stimulates an accelerated induction of the anagen phase, thereby stimulating hair growth. Because some hair-loss related conditions and diseases, such as Androgenetic Alopecia, are characterized by a shortened anagen phase duration (longer telogen phase and kenogen phase), these results indicate that ebastine is an effective drug for use in treatment of such hair-loss related conditions or diseases in humans.

### Clinical Signs

Clinical signs such as behavior were monitored throughout the study. No abnormal behavior was observed in any of the mice in either the ebastine treatment group or the nontreatment group. The patch area was assessed for signs of erythema using the modified Draize scoring system and dryness every other day throughout the study period. No signs of erythema or dryness of the skin were observed at the treatment site with daily application of the ebastine composition. These observations demonstrate the favorable cutaneous safety profile of topically applied ebastine compositions as described herein.

### Skin Uptake

At the end of the study period, the patch area was shaved and cleaned with waterless shampoo and 1×PBS. Three 6-millimeter punch biopsies were taken from within the patch area. Ebastine was extracted from each homogenized biopsy and analyzed by liquid chromatography with mass spectrometry (LC-MS/MS).

The average values of ebastine concentrations in skin for the three mice in the ebastine treatment group, are presented in Table 10. The results demonstrate that topical application of ebastine compositions as described herein allow significant penetration of ebastine into the skin. This suggests that ebastine is available for stimulating hair growth for treatment of a hair-loss related condition or disease.

TABLE 10
Ebastine Skin Penetration and Blood Plasma Penetration

| | Amount of Sample | Skin Concentration | Plasma |
|---|---|---|---|
| | applied daily | (μg/g) ± standard | Concentration |
| SAMPLE | (mg/cm²) | deviation (SD) | (ng/mL) ± SD |
| Ebastine | 2.5 mg/cm² | 40.4 +/− 40.2 | 2.18 +/− 0.63 |

### Blood Plasma Concentrations

At the end of the 5-week study period, blood was collected from each mouse in the ebastine treatment group by a terminal bleed. The plasma was analyzed by LC-MS/MS for ebastine by a method with a lower limit of quantification (LLOQ) of 0.5 ng/mL.

The average values for the three mice dosed with ebastine daily are presented in Table 10 above. The results demonstrate a minimal amount of ebastine in the blood, suggesting minimal systemic exposure. Although ebastine appears to be a safe and effective drug, minimal systemic exposure of ebastine indicates that potential adverse side effects due to systemic exposure can be avoided or reduced by topical application of ebastine compositions in comparison to oral or injected ebastine dosage forms.

### Embodiments

1. A topical pharmaceutical composition, comprising
ebastine, and
a solvent,
wherein the ebastine is dissolved in the composition at room temperature.

2. The topical pharmaceutical composition of embodiment 1,
wherein the solvent comprises a volatile solvent and a non-volatile solvent.

3. The topical pharmaceutical composition of embodiment 2, wherein the non-volatile solvent is evaporated to form a residual composition, and ebastine is dissolved in the residual composition.

4. The topical pharmaceutical composition of the combined or separate embodiments 2-3, wherein ebastine is more soluble at room temperature in the volatile solvent than in the non-volatile solvent.

5. The topical pharmaceutical composition of the combined or separate embodiments 2-3, wherein ebastine is more soluble at room temperature in the non-volatile solvent than in the volatile solvent.

6. The topical pharmaceutical composition of the combined or separate embodiments 2-5, wherein ebastine is more soluble at room temperature in the solvent than in either the volatile solvent or the non-volatile solvent.

7. The topical pharmaceutical composition of the combined or separate embodiments 2-6, wherein the volatile solvent is a monohydric aliphatic alcohol and the non-volatile solvent is an ester.

8. The topical pharmaceutical composition of embodiment 7, wherein the monohydric aliphatic alcohol is ethanol.

9. The topical pharmaceutical composition of the combined or separate embodiments 7-8, wherein the ester is selected from the group consisting of diethyl sebacate, dimethyl succinate, and ethyl acetate.

10. The topical pharmaceutical composition of the combined or separate embodiments 7-9, wherein the ratio of concentrations by weight of monohydric aliphatic alcohol to ester is in the range of 1:1 to 99:1.

11. The topical pharmaceutical composition of the combined or separate embodiments 7-10, wherein the w/w ratio of concentrations by weight of monohydric aliphatic alcohol to ester is in the range of 1:1 to 20:1.

12. The topical pharmaceutical composition of embodiment 7, wherein the monohydric aliphatic alcohol comprises 40 percent to 99 percent of the composition by weight.

13. The topical pharmaceutical composition of the combined or separate embodiments 2-6, wherein the ratio of concentrations by weight of the volatile solvent to the non-volatile solvent is in the range of 1:1 to 99:1.

14. The topical pharmaceutical composition of the combined or separate embodiments 2-6, wherein the w/w ratio of concentrations by weight of the volatile solvent alcohol to the non-volatile solvent is in the range of 1:1 to 20:1.

15. The topical pharmaceutical composition of the combined or separate embodiments 2-6, wherein the volatile solvent comprises 40 percent to 99 percent of the composition by weight.

16. The topical pharmaceutical composition of the combined or separate embodiments 1-15, wherein the solvent comprises a polyol.

17. The topical pharmaceutical composition of embodiment 16, wherein the polyol is propylene glycol.

18. The topical pharmaceutical composition of the combined or separate embodiments 1-17, wherein the ebastine is stable when stored in a sealed glass container for at least 2 months or longer at 40° C. in a dark environment.

19. The topical pharmaceutical composition of the combined or separate embodiments 1-18, wherein the solvent comprises water and a non-aqueous solvent.

20. The topical pharmaceutical composition of embodiment 19,
wherein the concentration of the non-aqueous solvent exceeds the concentration of water and
wherein the pH of the composition is in the range of 3 to 7.

21. The topical pharmaceutical composition of the combined or separate embodiments 1-20, wherein the concentration of ebastine in composition is in the range of 0.1 percent by weight to 5 percent by weight.

22. The topical pharmaceutical composition of the combined or separate embodiments 1-21, wherein the concentration of ebastine in the composition is about 2.0 percent w/w by weight.

23. The topical pharmaceutical composition of the combined or separate embodiments 1-22, wherein the composition is non-aqueous.

24. The topical pharmaceutical composition of the combined or separate embodiments 1-23, wherein the composition does not comprise an active pharmaceutical ingredient other than ebastine.

25. A method for the treatment of a hair-loss related condition or disease comprising topically applying a composition comprising ebastine dissolved in a solvent to a target skin surface of mammalian body at least once daily for a period of at least one month.

26. The method of embodiment 25, wherein the hair-loss related condition or disease is selected from the group consisting of scarring alopecia, non-scarring alopecia, androgenetic alopecia, and alopecia areata.

27. A method for the treatment of a dermatological disorder that benefits from antihistamine activity comprising topically applying a composition comprising ebastine dissolved in a solvent to a target surface of a mammalian body at least once daily for a period of at least one week.

28. The method of the combined or separate embodiments 25-27, wherein the solvent is non-aqueous or comprises a volatile solvent and a non-volatile solvent.

29. The method of the combined or separate embodiments 25-28, wherein the target skin surface is a scalp.

30. The method of the combined or separate embodiments 25-29, wherein the daily dosage of ebastine is in the range of 1.0 mg/cm² to 10 mg/cm².

31. The method of embodiment 30, wherein the daily dosage of ebastine is about 2.5 mg/cm².

32. The method of the combined or separate embodiments 25-31, wherein the composition does not comprise an active pharmaceutical ingredient other than ebastine.

33. A method for preparing a topical pharmaceutical composition, the method comprising (i) combining ebastine in a solvent to form a mixture, and (ii) agitating the mixture from (i) to form a solution in which the ebastine is dissolved.

34. A kit for the delivery of a topical pharmaceutical composition comprising
a container closure,
a topical pharmaceutical composition comprising ebastine dissolved in a solvent, and instructions for use,
wherein instructions for use comprises instructions to apply the composition to a skin surface, such as the scalp, of a patient.

35. The kit of embodiment 34,
wherein the container closure comprises a nozzle capable of releasing the composition.

36. The kit of the combined or separate embodiments 34-35, wherein the skin surface is the scalp and the instructions further specify applying the composition to the skin surface once daily.

It is claimed:

1. A topical pharmaceutical composition, comprising:
ebastine, and
a solvent,
wherein the ebastine is dissolved in the composition at room temperature.

2. The topical pharmaceutical composition of claim 1,
wherein the solvent comprises a volatile solvent and a non-volatile solvent.

3. The topical pharmaceutical composition of claim 2, wherein the non-volatile solvent is evaporated to form a residual composition, and ebastine is dissolved in the residual composition.

4. The topical pharmaceutical composition of claim 2, wherein ebastine is more soluble at room temperature in the volatile solvent than in the non-volatile solvent.

5. The topical pharmaceutical composition of claim 2, wherein ebastine is more soluble at room temperature in the non-volatile solvent than in the volatile solvent.

6. The topical pharmaceutical composition of claim 2, wherein ebastine is more soluble at room temperature in the solvent than in either the volatile solvent or the non-volatile solvent.

7. The topical pharmaceutical composition of claim 2, wherein the volatile solvent is a monohydric aliphatic alcohol and the non-volatile solvent is an ester.

8. The topical pharmaceutical composition of claim 7, wherein the monohydric aliphatic alcohol is ethanol.

9. The topical pharmaceutical composition of claim 7, wherein the ester is selected from the group consisting of diethyl sebacate, dimethyl succinate, and ethyl acetate.

10. The topical pharmaceutical composition of claim 7, wherein the ratio of concentrations by weight of monohydric aliphatic alcohol to ester is in the range of 1:1 to 99:1.

11. The topical pharmaceutical composition of claim 7, wherein the monohydric aliphatic alcohol comprises 40 percent to 99 percent of the composition by weight.

12. The topical pharmaceutical composition of claim 1, wherein the solvent comprises a polyol.

13. The topical pharmaceutical composition of claim 12, wherein the polyol is propylene glycol.

14. The topical pharmaceutical composition of claim 1, wherein the ebastine is stable when stored in a sealed glass container for at least 2 months or longer at 40° C. in a dark environment.

15. The topical pharmaceutical composition of claim 1,
wherein the solvent comprises
a concentration of water and
a concentration of a non-aqueous solvent,
wherein the concentration of the non-aqueous solvent exceeds the concentration of water, and
wherein the pH of the composition is in the range of 3 to 7.

16. The topical pharmaceutical composition of claim 1, wherein the composition is non-aqueous.

17. The topical pharmaceutical composition of claim 1, wherein the composition does not comprise an active pharmaceutical ingredient other than ebastine.

18. A method for the treatment of a hair-loss related condition or disease, comprising: topically applying a composition comprising ebastine dissolved in a solvent to the scalp of a human patient at least once daily for a period of at least one month.

19. The method of claim 18, wherein the hair-loss related condition or disease is selected from the group consisting of scarring alopecia, non-scarring alopecia, androgenetic alopecia, and alopecia areata .

20. The method of claim 18, wherein the daily dosage of ebastine is in the range of 1.0 mg/cm² to 10 mg/cm².

21. The method of claim 18, wherein the composition does not comprise an active pharmaceutical ingredient other than ebastine.

22. A kit for the delivery of a topical pharmaceutical composition, comprising:
a container closure,
a topical pharmaceutical composition comprising ebastine dissolved in a solvent, and instructions for use,
wherein the instructions for use comprises instructions to apply the composition to the scalp of a patient.
